(19) United States
(12) Patent Application Publication
(10) Pub. No.: US 2009/0124787 A1
(43) Pub. Date: May 14, 2009

(54) Method for Producing Polyhydroxyalkanoates

(76) Inventors: Peter Preishuber-Pflugl, Ludwigshafen (DE); Ferenc Molnar, Speyer (DE)

Correspondence Address: CONNOLLY BOVE LODGE & HUTZ, LLP, P O BOX 2207, WILMINGTON, DE, 19899, US

(21) Appl. No.: 11/911,297

(22) PCT Filed: Apr. 11, 2006
(86) PCT No.: PCT/EP06/61501

§ 371 Date: Oct. 11, 2007

(30) Foreign Application Priority Data
Apr. 12, 2005 (DE) 10 2005 017 049.8

(51) Int. Cl. C08G 63/82; C07D 305/12

(52) U.S. Cl. 528/355; 549/328

(57) ABSTRACT

The invention relates to a process for preparing polyhydroxyalkanoates by polymerization of lactones of the general formula I,

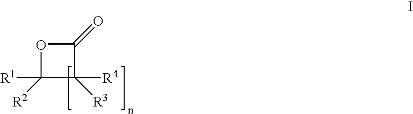

where the substituents and the index n have the meanings given in the description, in the presence of at least one catalyst of the formula (II) L_{I}M^{a}X^{a}ₘ, where the substituents and indices have the meanings given in the description. The invention further relates to poly-3-hydroxybutyrates which have a novel property profile and are obtainable for the first time by means of this process, and also biodegradable polyester mixtures based on these poly-3-hydroxybutyrates.

The invention relates to a process for preparing polyhydroxyalkanoates by polymerization of lactones of the general formula I,

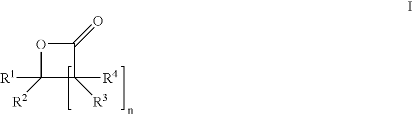

where the substituents and the index n have the following meanings:
n is from 1 to 4;
R¹, R², R³, R⁴ are each, independently of one another, hydrogen, C₁-C₁₂-alkyl, C₂-C₈-alkenyl, C₃-C₈-cycloalkyl, C₆-C₁₂-aryl, C₆-C₁₂-aryl-C₁-C₃-alkyl, halogen, nitro, C₁-C₆-alkoxy, C₆-C₁₂-aryloxy, amino, C₁-C₆-alkylamino, di(C₁-C₆-alkyl)amino, di(C₁-C₆-alkyl)phosphino, C₁-C₆-alkylsulfinyl, C₁-C₆-alkylsulfonyl;
two radicals R¹ to R⁴ located on adjacent ring carbons together form C₁-C₅-alkylene; where R¹ to R⁴ may in turn be substituted by R^{x} and R^{x} represents from one to three radicals selected from among halogen, cyano, nitro, C₁-C₆-alkoxy, C₁-C₆-alkylthio, di(C₁-C₆-alkyl)amino, C₆-C₁₂-aryloxy, C₆-C₁₂-aryl-C₁-C₃-alkoxy, C₁-C₆-alkoxycarbonyl, C₆-C₁₂-aryloxycarbonyl, C₆-C₁₂-aryl-C₁-C₃-alkoxycarbonyl, C₁-C₆-alkylcarbonyl, C₆-C₁₂-arylcarbonyl, C₆-C₁₂-aryl-C₁-C₃-alkylcarbonyl, C₁-C₆-alkylsulfinyl, C₆-C₁₂-arylsulfinyl, C₆-C₁₂-aryl-C₁-C₃-alkylsulfinyl, C₁-C₆-alkylsulfonyl, C₆-C₁₂-arylsulfonyl, C₆-C₁₂-aryl-C₁-C₃-alkylsulfonyl;
i) in the presence of at least one catalyst of the formula (II) L_{I}M^{a}X^{a}ₘ, where the substituents and indices have the following meanings:
M^{a} is a metal selected from the group consisting of Cr, Mo and W,
X^{a} is an anion selected from the group consisting of halide, sulfate, sulfite, nitrate, nitrite, carboxylate, hydroxide, alkoxide, thiolate, phosphate, sulfonate, borate, phenoxide, antimonate, cobaltate and ferrate,
L is a ligand selected from the group consisting of imine, amine, phosphane, ylide, carbonyl, nitrile, ester, ether, sulfide, amide, cyclopentadienyl, ansa compounds, alkoxide, phenoxide, carboxylate, thiolate, imide, sulfonate, porphyrin, phthalocyanine, oxazoline, salen and Schiff base compounds, with different ligands L also being able to be joined to one another and the ligands L being able to be substituted,
l, m are integers from 1 to 10 which are selected so that the compound of the general formula II is uncharged;
ii) if appropriate in the presence of an activator compound B selected from among unsubstituted or substituted pyridines, imidazoles, triazoles, carbenes, phosphines and ionic compounds of the general formula (III) X^{b}ₒYₚ, where the substituents and indices have the following meanings:
X^{b} is a cation selected from the group consisting of H⁺, Na⁺, K⁺, Li⁺, Mg²⁺, Ca²⁺, Al³⁺, NR₄⁺, pyridinium, imidazolium, PR₄⁺, AsR₄⁺and N[PR₃)]₂⁺, where R is hydrogen, C₁-C₆-alkyl or C₆-C₁₂₋aryl;
Y is an anion selected from the group consisting of halide, carboxylate, dicarboxylate, tricarboxylate, polycarboxylate, sulfonate, sulfonyl, sulfate, sulfinylate, phosphate, phosphite, hydroxide, alkoxide, dialkoxide, trialkoxide, polyalkoxide, thiolate, acyl, carbonate, carbamate, antimonate and borate;
o, p are integers from 1 to 10 000 which are selected so that the compound of the general formula III is uncharged;
iii) if appropriate in the presence of a Lewis acid C of the general formula (IV) L_{q}M^{c}X^{c}ᵣ, where the substituents and indices have the following meanings:
M^{c} is a metal selected from the group consisting of Mg, Ca, Sc, Y, rare earth elements, Ti, V, Mn, Fe, Co, Ni, Cu, Zn, Al, Ga, Zr, Nb, Ru, Rh, Pd, Ag, Cd, In, Hf, Ta, Re, Os, Ir, Pt, Au, Hg, Tl and Pb,
X^{c} is an anion selected from the group consisting of halide, sulfate, sulfite, nitrate, nitrite, carboxylate, thiolate, phosphate, sulfonate, borate, hydroxide, alkoxide, phenoxide, antimonate, cobaltate and ferrate,
L is a ligand selected from the group consisting of imine, amine, phosphane, ylide, carbonyl, nitrile, ester, ether, sulfide, amide, cyclopentadienyl, ansa compounds, alkoxide, phenoxide, carboxylate, thiolate, imide, sulfonate, porphyrin, phthalocyanine, oxazoline, salen and Schiff base compounds, with different ligands L also being able to be joined to one another and the ligands L being able to be substituted,
q, r are integers from 1 to 10 which are selected so that the compound of the general formula IV is uncharged.

The invention further relates to poly-3-hydroxybutyrates which have a novel property profile and are obtainable by the above-described process, and also a biodegradable polyester mixture comprising poly-3-hydroxybutyrate.

Polyhydroxyalkanoates are interesting polymers which are suitable for numerous polymer applications. Stereoregular polyhydroxyalkanoates which can be obtained by polymerization of chiral lactones are of particular interest, since the properties can be tailored via the steric structure of the polymer.

An example of a polyester which can be prepared with different steric structures and thus different properties is poly(3-hydroxybutyrate).

Highly isotactic poly(3-hydroxybutyrate), which can be prepared biochemically, has a melting point of 170-180° C. Thermoplastic processing has to be carried out at temperatures at which thermal degradation of the polymer commences. Atactic poly(3-hydroxybutyrate), on the other hand, has no melting point but only a glass transition at about 5° C. This polymer is therefore not of interest for thermoplastic properties (cf. WO-A 94/00506).

U.S. Pat. No. 5,440,007 and U.S. Pat. No. 6,545,112 describe the preparation of poly-beta-propio esters having a preferably syndiotactic structure. Organometallic tin compounds are used as catalysts for this purpose. The polymer has a melting point of about 60° C.

Macromolecules 1988, 21, 2657, 163, describes the polymerization of racemic beta-butyrolactone using trialkylaluminum/water systems to form isotactic poly(3-hydroxybutyrate). Crystalline polymers having a melting point in the range from 159° C. to 163° C. are obtained.

Macromolecules 1996, 29, 8683, and Macromolecules 1998, 31, 3473, disclose the polymerization of racemic beta-butyrolactone by means of aluminoxane to form isotactic poly(3-hydroxybutyrate). The polymer was fractionated and the highest-melting polymer has a melting point of 166° C.

In the abovementioned aluminum-catalyzed processes, very high concentrations of catalyst are necessary in order to achieve a good activity. As a result, very high proportions of inorganic compounds are obtained in the polymer, so that complicated and costly purification of the polymer is necessary. In addition, the cost of the polymerization catalysts is very high, which ultimately leads to very high production costs and makes the system uninteresting from an economic point of view.

Finally, the known processes of the prior art starting out from chiral lactones give either atactic or syndiotactic polyhydroxyalkanoate having a very low melting point or isotactic polyhydroxyalkanoate having a melting point which is too high, nearly at the temperature at which thermal decomposition of the polymer commences.

It was therefore an object of the invention to provide a process which does not have the abovementioned disadvantages and makes the synthesis of preferably isotactic polyhydroxyalkanoate (proportion of isotactic diads of from 55 to 90%) having a high molecular weight possible.

The process described at the outset surprisingly achieves this object.

The process of the invention will be described in more detail below.

Lactones in particular are suitable as starting material for preparing the polyhydroxyalkanoates.

The lactones are commercially available or can be obtained in a manner known per se. Some methods of synthesizing lactones are described in Tetrahedron 1999, 55, 6403, or Chem. Eur. J. 2003, 9, 1273.

The synthesis of stereoregular polyhydroxyalkanoates correspondingly starts out from chiral lactones. In these cases, it is possible to use racemic lactone mixtures (without an enantiomeric excess) or enantiomerically enriched lactones (R or S enantiomer in excess).

Preference is given to using a racemic lactone mixture.

Lactones which can be used are, in particular, β-lactones, γ-lactones, δ-lactones and ε-lactones of the general formula I,

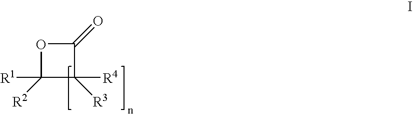

where the substituents and the index n have the following meanings:
n is from 1 to 4;
R¹, R², R³, R⁴ are each, independently of one another, hydrogen, C₁-C₁₂-alkyl, C₂-C₈-alkenyl, C₃-C₈-cycloalkyl, C₆-C₁₂-aryl, C₆-C₁₂-aryl-C₁-C₃-alkyl, halogen, nitro, C₁-C₆-alkoxy, C₆-C₁₂-aryloxy, amino, C₁-C₆-alkylamino, di(C₁-C₆-alkyl)amino, di(C₁-C₆-alkyl)phosphino, C₁-C₆-alkylsulfinyl, C₁-C₆-alkylsulfonyl;
two radicals R¹ to R⁴ located on adjacent ring carbons together form C₁-C₅-alkylene; where R¹ to R⁴ may in turn be substituted by R^{x} and R^{x} represents from one to three radicals selected from among halogen, cyano, nitro, C₁-C₆-alkoxy, C₁-C₆-alkylthio, di(C₁-C₆-alkyl)amino, C₆-C₁₂-aryloxy, C₆-C₁₂-aryl-C₁-C₃-alkoxy, C₁-C₆-alkoxycarbonyl, C₆-C₁₂-aryloxycarbonyl, C₆-C₁₂-aryl-C₁-C₃-alkoxycarbonyl, C₁-C₆-alkylcarbonyl, C₆-C₁₂-arylcarbonyl, C₆-C₁₂-aryl-C₁-C₃-alkylcarbonyl, C₁-C₆-alkylsulfinyl, C₆-C₁₂-arylsulfinyl, C₆-C₁₂-aryl-C₁-C₃-alkylsulfinyl, C₁-C₆-alkylsulfonyl, C₆-C₁₂-arylsulfonyl, C₆-C₁₂-aryl-C₁-C₃-alkylsulfonyl.

Suitable radicals R¹ to R⁴ are, for example, C₁₋₁₂-alkyl such as methyl, ethyl, i- or n-propyl, i-, n- or t-butyl, n-pentyl or n-hexyl; C₂₋₈-alkenyl such as prop-1-enyl or but-2-enyl; C₃₋₈-cycloalkyl such as cyclopropyl, cyclobutyl, cyclopentyl or cyclohexyl; C₆₋₁₂-aryl such as phenyl or naphthyl, and C₆-C₁₂-aryl-C₁-C₃-alkyl such as benzyl. The radicals R¹ to R⁴ can also be bound to the lactone ring via a heteroatom, e.g. the radicals: chloride, bromide, fluoride, dimethylamino, methoxy or phenoxy. Here, two radicals R located on different carbon atoms of the lactone ring can be joined to one another and form a C₁-C₅-alkylene radical such as an ethylene or propylene radical. The radicals R¹ to R⁴ may in turn be substituted, for example by the groups chlorine, bromine, methyl, methoxy, cyano, methoxycarbonyl.

Examples of suitable lactones are:

β-lactones: β-propiolactone, β-butyrolactone, diketene, 4-ethyloxetan-2-one, 4-propyloxetan-2-one, 4-isopropyloxetan-2-one, 4-phenyloxetan-2-one, 4,4-dimethyloxetan-2-one, 4,4-diethyloxetan-2-one, 4,4-diphenyloxetan-2-one, 3,4-dimethyloxetan-2-one, 3,4-diphenyloxetan-2-one, 7-oxabicyclo[4.2.0]octan-8-one; γ-lactones: γ-butyrolactone, γ-valerolactone, 3-methyldihydrofuran-2-one, 3,4-dimethyldihydrofuran-2-one; δ-lactones: δ-valerolactone, 5,6-dimethyltetrahydropyran-2-one; ε-lactones: ε-caprolactone, ε-4-methylcaprolactone.

Particular preference is given to β-butyrolactone.

The lactone can be prepurified. One possibility here is removing the water by addition of molecular sieves or distillation over calcium hydride. Further prepurification, e.g. treatment with basic compounds to remove acid, can in general be dispensed with.

As catalyst (in the claims, cf. component i), it is possible to use compounds of the formula (II) L_{I}M^{a}X^{a}ₘ, where the substituents and indices have the following meanings:
M^{a} is a metal selected from the group consisting of Cr, Mo and W,
X^{a} is an anion selected from the group consisting of halide, sulfate, sulfite, nitrate, nitrite, carboxylate, thiolate, phosphate, sulfonate, borate, hydroxide, alkoxide, phenoxide, antimonate, cobaltate and ferrate,
L is a ligand selected from the group consisting of imine, amine, phosphane, ylide, carbonyl, nitrile, ester, ether, sulfide, amide, cyclopentadienyl, ansa compounds, alkoxide, phenoxide, carboxylate, thiolate, imide, sulfonate, porphyrin, phthalocyanine, oxazoline, salen and Schiff base compounds, with different ligands L also being able to be joined to one another and the ligands L being able to be substituted,
I, m are integers from 1 to 10 which are selected so that the compound of the general formula II is uncharged.

These compounds either have coordinative undersaturation a priori, or they can (reversibly) eliminate a ligand, solvent or water under the reaction conditions of the polymerization according to the invention so that coordinative undersaturation is obtained under the reaction conditions. The catalysts preferably bind ligands which do not participate in the reaction but exert a controlling influence on the polymerization. The ligand-metal units can be chiral.

The metal M is preferably chromium.

Preferred anions X are chloride, bromide, iodide, tetrafluoroborate, hexafluoroantimonate, hexafluorophosphate, sulfonate, hydroxide, carboxylate and alkoxide, for example dinitrophenoxide.

Preferred ligands are porphyrin, phthalocyanine and salen, particularly preferably salen.

Salen structures can be prepared by condensation of diamines and aldehydes or ketones. The carbonyl compounds can be identical or different here.

Salens have the general formula

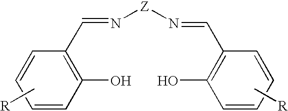

Examples of diamines Z(NH₂)₂ which are suitable for building salens are:

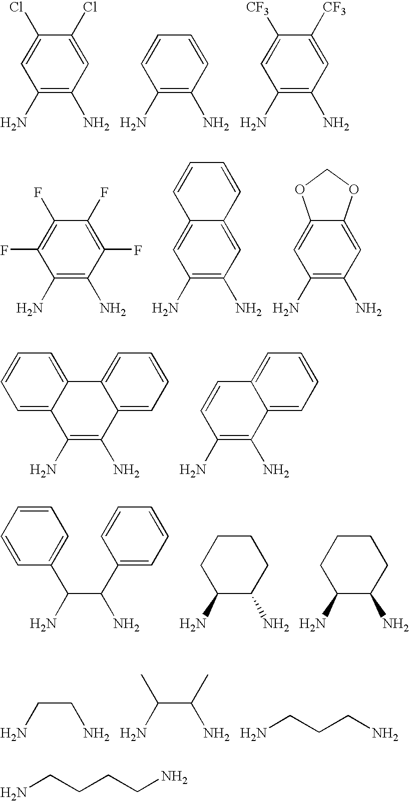

Examples of aldehydes are:

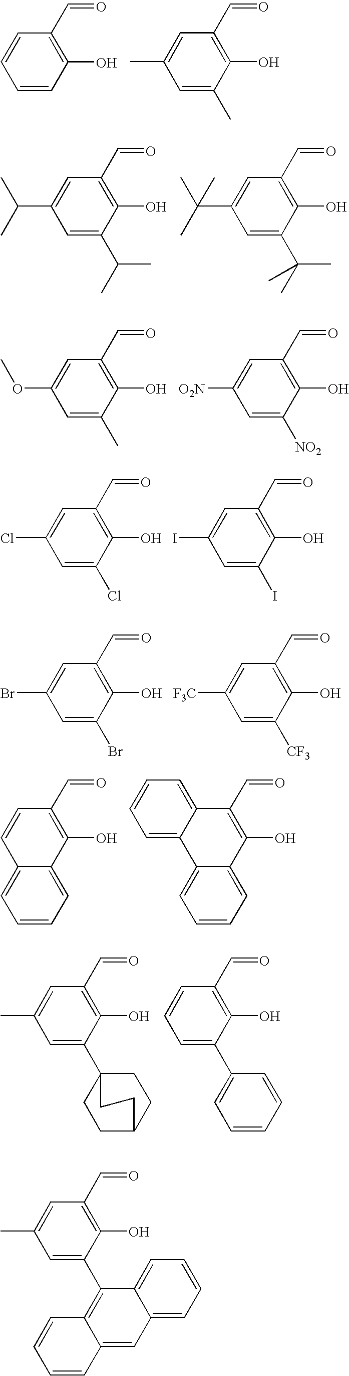

Further salen ligands can be prepared by condensation of ketones and diamines.

The ligands can be used in their enantiomerically pure form.

Suitable salens are, for example,

(1R,2R)-[1,2-cyclohexanediamino-N,N′-bis-3,5-di-t-butylsalicylidene],

(1S,2S)-[1,2-cyclohexanediamino-N,N′-bis-3,5-diiodosalicylidene],

1,2-phenylenediamino-N,N′-bis-3,5-di-t-butylsalicylidene] and

[4,5-dichloro-1,2-phenylenediamino-N,N′-bis-3,5-di-t-butylsalicylidene]

Oxazolines preferred as ligands L are, for example,

1,2-bis(2,4-dimethyl-2-oxazolin-2-yl)ethane,

(S,S)-2,2′-bis(4-benzyl-2-oxazoline),

(S,S)-2,2′-(2,6-pyridinediyl)bis(4-isopropyl-2-oxazoline),

(S,S)-(−)-2,2′-(dimethylmethylene)bis(4-tert-butyl-2-oxazoline), and

(4R,5S,4′R,5′S)-2,2′-methylenebis(4,5-diphenyl-2-oxazoline).

Schiff base compounds, also referred to as azomethines, condensation products of aldehydes or other carbonyl compounds and primary amines, preferred as ligands L are, for example,

(1R,2S)-[1-[(3,5-di-tert-butyl-2-hydroxybenzylidene)amino]indan-2-ol],

(1R,2S)-[1-[(3-adamantyl-2-hydroxy-5-methylbenzylidene)amino]indan-2-ol],

(1S,2R)-[1-[(3-adamantyl-2-hydroxy-5-methylbenzylidene)amino]indan-2-ol], and

(1R,2S)-[1-[(3-adamantyl-2-hydroxy-5-methylbenzylidene)amino]-1,2-diphenylethan-2-ol.

Phosphanes preferred as ligands L are, for example:

(2S,4S)-(−)-(diphenylphosphino)-2-(diphenylphosphinomethyl)pyrrolidine,

(R)-(+)-2,2′-bis(diphenylphosphino)-1,1′-binaphthyl (Binap),

R-(+)-1,2-bis(diphenylphosphino)propane,

(4R,5R)-(−)-o-isopropylidene-2,3-dihydroxy-1,4-bis(diphenylphosphino)butane (Diop),

(1S,2S)-(+)-1,2-bis(diphenylphosphinomethyl)cyclohexane,

(−)-(R)-N,N-dimethyl-1-[(S)-1′,2-bis(diphenylphosphino)ferrocenyl]ethylamine,

(2R,3R)-(+)-bis(diphenylphosphino)butane,

(+)-1,2-bis[(2S,5S)-2,5-dimethylphospholano]benzene,

(S)-1((R)-1′,2-bis(diphenylphosphino)ferrocenyl)ethanol,

(R)-(−)-1-[(S)-2-(diphenylphosphino)ferrocenyl]ethyldicyclohexylphosphine, and

(1S,2S)-(+)-1,2-bis[(n-diphenylphosphino)amino]cyclohexane).

The ansa compounds (also referred to as cyclophanes) mentioned for the ligand L are aromatic compounds which are bridged by an aliphatic chain.

In addition, ethylenebis(4,5,6,7-tetrahydro-1-indenyl), binaphthol and amino acids are also suitable as ligands L.

Further suitable ligands are known to those skilled in the art from catalysis using homogeneous organic metal compounds.

Preferred catalysts of the formula II are, for example,

(1R,2R)-[1,2-cyclohexanediamino-N,N′-bis-3,5-di-t-butylsalicylidene]chromium(III) chloride,

[1,2-phenylenediamino-N,N′-bis-3,5-di-t-butylsalicylidene]chromium(III) chloride,

[4,5-dichloro-1,2-phenylenediamino-N,N′-bis-3,5-di-t-butylsalicylidene]chromium(III) chloride

and (1R,2S)-[1-[(3-adamantyl-2-hydroxy-5-methylbenzylidene)amino]indan-2-ol]chromium(III) chloride.

The preparation of the catalysts II is known to those skilled in the art and is also described in WO-A 00/09463. Moreover, numerous compounds II are also commercially available.

Of course, it is also possible to use mixtures of various catalysts. The catalysts can be mononuclear or multinuclear.

The catalyst can further comprise an activator compound B which is selected from among unsubstituted or substituted pyridines, imidazoles, triazoles, carbenes, phosphines and ionic compounds of the general formula (III) X^{b}ₒYₚ, where the substituents and indices have the following meanings:
X^{b} is a cation selected from the group consisting of H⁺, Na⁺, K⁺, Li⁺, Mg²⁺, Ca²⁺, Al³⁺, NR₄⁺, pyridinium, imidazolium, PR₄⁺, AsR₄⁺ and N[PR₃)]₂⁺, where R is hydrogen, C₁-C₆-alkyl or C₆-C₁₂-aryl;
Y is an anion selected from the group consisting of halide, carboxylate, dicarboxylate, tricarboxylate, polycarboxylate, sulfonate, sulfonyl, sulfate, sulfinylate, phosphate, phosphite, hydroxide, alkoxide, dialkoxide, trialkoxide, polyalkoxide, sulfide, acyl, carbonate, carbamate, antimonate and borate;
o, p are integers from 1 to 10 000 which are selected so that the compound of the general formula III is uncharged.

Preference is given to X being NR₄⁺ or pyridinium and Y being chloride, carboxylate, dicarboxylate or tricarboxylate. A mixture of various activator compounds III can also be added. The use of polyfunctional carboxylates leads to branched structures and a higher molecular weight.

If appropriate, a Lewis acid C of the general formula L_{q}M^{c}X^{c}ᵣ (IV), where the substituents and indices have the following meanings:
M^{c} is a metal selected from the group consisting of Mg, Ca, Sc, Y, rare earth elements, Ti, V, Mn, Fe, Co, Ni, Cu, Zn, Al, Ga, Zr, Nb, Ru, Rh, Pd, Ag, Cd, In, Hf, Ta, Re, Os, Ir, Pt, Au, Hg, Tl and Pb,
X^{c} is an anion selected from the group consisting of halide, sulfate, sulfite, nitrate, nitrite, carboxylate, hydroxide, alkoxide, thiolate, phosphate, sulfonate, borate, phenoxide, antimonate, cobaltate and ferrate,
L is a ligand selected from the group consisting of imine, amine, phosphane, ylide, carbonyl, nitrile, ester, ether, sulfide, amide, cyclopentadienyl, ansa compounds, alkoxide, phenoxide, carboxylate, thiolate, imide, sulfonate, porphyrin, phthalocyanine, oxazoline, salen and Schiff base compounds, with different ligands L also being able to be joined to one another and the ligands L being able to be substituted,
q, r are integers from 1 to 10 which are selected so that the compound of the general formula IV is uncharged,

can also be added to the catalyst system.

The amount of catalyst (II) in the reaction mixture is usually in the range from 0.0001 to 100 mol %, preferably from 0.001 to 5 mol %, particularly preferably from 0.001 to 0.5 mol %, based on the amount of lactone used.

The amount of activator compound (III) in the reaction mixture is usually in the range from 0.0001 to 100 mol %, preferably from 0.001 to 5 mol %, particularly preferably from 0.001 to 0.5 mol %, based on the amount of lactone used.

The amount of Lewis acid C (IV) in the reaction mixture is usually in the range from 0.0001 to 100 mol %, preferably from 0.001 to 5 mol %, particularly preferably from 0.001 to 0.5 mol %, based on the amount of lactone used.

The compounds II and III are used in a ratio of from 1:10 000 to 10 000:1, preferably from 1:100 to 10 000: 1, particularly preferably from 1:1 to 1000:1.

The compounds II and IV are used in a ratio of from 1:10 000 to 10 000:1, preferably from 1: 1000 to 1000: 1, particularly preferably from 1:100 to 100:1.

The lactones can be copolymerized with further reactive monomers, for example the following cyclic compounds: lactides, glycosides, lactams, dioxepanediones, epoxides, aziridines, carbonates or anhydrides.

Copolymers comprising 0.01-99.9% of comonomer, preferably 1-30% and particularly preferably 5-20% of comonomer, can be prepared by means of the process of the invention.

The molecular weights of the polyesters are in the range from 500 to 5 000 000, preferably from 50 000 to 3 000 000.

The molar masses of the polymers can be regulated by means of suitable compounds. Suitable compounds for this purpose are, for example, alcohols, diols, amines, carboxylic acids.

Low molecular weight polyhydroxyalkanoates can be used as macromonomers. Higher molecular weights can be built up by means of, for example, chain extenders such as isocyanates.

Furthermore, branched or crosslinked polymers can be prepared by means of the process of the invention. For example, a crosslinked polymer can be obtained by use of polyfunctional epoxides or a branched polymer can be obtained using polyfunctional ammonium carboxylate as activator compound III (cf. WO-A 94/00506).

The (polymerization) process of the invention can be carried out with or without addition of solvents. Possible solvents are all customary solvents. The polymerization is preferably carried out without solvent.

The polymerization can be carried out as a melt polymerization. Here, the polymerization is carried out above the softening point of the polymer.

The polymerization can be carried out as a solution polymerization. Here, a solvent in which the polymer is soluble under polymerization conditions is chosen.

The polymerization can be carried out as a precipitation polymerization. Here, a solvent in which the monomer is soluble and the polymer is insoluble under polymerization conditions is chosen.

The polymerization can be carried out in supercritical gases or ionic liquids.

The process of the invention is generally carried out at temperatures of from −100 to 250° C., preferably 0-180° C., particularly preferably 60-140° C.

The process can be carried out at superatmospheric pressure, but is preferably carried out at atmospheric pressure. As pressurizing gas, it is possible to use, for example, nitrogen, argon, carbon monoxide, carbon dioxide, ethylene or propylene.

The process of the invention can be carried out either batchwise (discontinuously) or continuously.

The catalyst II and, if appropriate, the compounds III and IV can be removed from the polymer after the polymerization. It can sometimes be advisable to remove only the compounds II to IV only partially and to remove only one or two of the compounds from the polymer.

A possibility here is to select one or more of the compounds so that after the reaction they go over into a phase which separates from the polymer. An example of removal of a catalyst by means of a fluorine-comprising phase has been described in J. Am. Chem. Soc., 1998, 120, 3133. Catalysts II having fluorine-comprising side groups can, for example, be used for this purpose.

A further possibility is to immobilize one or more of the compounds I to III on a support material in order to make simpler removal of the catalyst system from the polymer possible. Suitable support materials can be silica, aluminum oxide, activated carbon or crosslinked polystyrene.

The support material can be selected so that it is soluble in the monomer or polymer during the reaction and becomes insoluble when the temperature is changed.

The compounds I to III can be bound to the support material by means of ionic interactions or covalently. As an alternative, the compounds I to III can be bound to the support material only after the polymerization and removed in this way from the polymer.

The compounds I to III can finally be removed again from the support material and be employed in a renewed reaction. A possibility which may be mentioned here is ionic interactions of the compounds I to III with ion exchangers.

The polymers obtained by these processes can be used in pure form or as a blend.

In general, blends comprise from 1 to 99% by weight of the polymer prepared by the process of the invention, preferably 5-85% by weight and particularly preferably 20-70% by weight.

The polymer can, for example, be blended with various polyhydroxyalkanoates. Polyhydroxyalkanoates suitable for this purpose are, in particular, biochemically prepared or syndiotactic polyhydroxyalkanoates. Further possible blend materials are, for example, polyethers, cellulose esters, starch, modified starch, polyesters, polyester ethers, polyvinyl alcohol or polyacrylates. Preferred polyethers are polyethylene oxide and polypropylene oxide. Preferred polyesters are aliphatic, aromatic or aliphatic-aromatic polyesters. Particularly preferred polyesters are polylactic acid and highly isotactic poly(3-hydroxybutyrate) and copolymers thereof.

The polymers obtained by means of this process can be used for the production of biodegradable materials.

In an advantageous embodiment of the process, the lactone is prepared from oxirane (epoxide) and carbon monoxide in the presence of a carbonylation catalyst V in a preceding step and is converted into the polyhydroxyalkanoate without intermediate isolation of the lactone.

Furthermore, one or more compounds of the compounds used in the polymerization step selected from the group consisting of catalyst II, activator compound III and Lewis acid IV which can, as defined at the outset, be present in the lactone preparation can be present in addition to the carbonylation catalyst V or alternatively can be added after lactone formation.

The transition metal complexes used as carbonylation catalyst V are uncharged (Vn) or anionic (Va) complexes.

Suitable uncharged complexes Vn are all uncharged complexes based on the metals of groups 5 to 11 of the PTE in which the central metal formally bears a charge of zero. Suitable metals are, for example, V, Ru, Cr, Mo, W, Mn, Re, Fe, Os, Co, Ir, Rh and Ni. Particular preference is given to Re, Co, Ru, Rh, Fe, Ni, Mn, Mo, W or mixtures thereof, in particular Co.

In the uncharged complex Vn, the ligands are generally present as uncharged ligands. The number of ligands depends on the respective metal and is determined by the coordinative saturation of the transition metal in the ground state. Suitable uncharged ligands are, for example, the carbon monoxide, nitro, nitroso, carbonate, ether, sulfoxide, amide, nitrile, phosphite or phosphine ligands. These ligands are generally coordinated to the transition metal via a free electron pair. Preference is given to using carbon monoxide as ligand.

Different ligands can also be present side by side in an uncharged complex Vn, as in Co₂(CO)₆(PMe₂Ph)₂. Preferred complexes A are: Co₂(CO)₈, Ru₃(CO)₁₂, Rh₄(CO)₁₂, Rh₆(CO)₁₆, Co₄(CO)₁₂, Fe₂(CO)₁₀, Fe₂(CO)₉, Ni(CO)₄, Mn₂(CO)₁₀, Mo(CO)₆ and W(CO)₆ or mixtures thereof. Particular preference is given to Ru₃(CO)₁₂, Co₄(CO)₁₂, Co(CO)₃(NO), Ni(CO)₄ and Mn₂(CO)₁₀, in particular Co₂(CO)₈.

The preparation of the uncharged complexes Vn is known to those skilled in the art and is described, for example, in F. G. Stone, E. W. Abel and G. Wilkinson, “Comprehensive Organometallic Chemistry—The Synthesis, Reactions and Structures of Organometallic Compounds”, Pergamon Press, Oxford, 1982, for example in Vol. 5. Such complexes can also be generated in situ, cf. EP-A 0 577 206. Moreover, such complexes are also commercially available.

Anionic complexes Va in the context of the present invention are compounds in which at least one central metal or ligand unit formally has a negative charge. Suitable anionic complexes Va have a central metal (designated as M_{β} in the formula (Va) below) from groups 5 to 11, preferably from groups 8 to 10, of the PTE. Possible metals are, for example, Co, Fe, Rh and Ru, preferably Co, Ru and Rh. Co is particularly preferred.

In the anionic complex Va, the ligands are usually likewise present as uncharged ligands. The number of ligands depends on the respective metal and is determined by the coordinative saturation of the transition metal in the ground state. Suitable uncharged ligands are, for example, the carbon monoxide, nitro, nitroso, carbonate, ether, sulfoxide, amide, nitrile, phosphite or phosphine ligands. These ligands are generally coordinated to the transition metal via a free electron pair. Preference is given to using carbon monoxide as ligand.

Different ligands can also be present side by side in the anionic complex Va, for example [P(Ph)₃]Co(−1)(CO)₃, [P(Me₂Ph)]Co(−1)(CO)₃, Co(−1)(CO)₃(CNPh). These compounds, too, can be generated in situ.

As carbonylation catalyst, preference is given to using transition metal complexes of the general formula (Va)

(M_{α}⁽ⁿ⁺⁾)ₘ[M_{β}(L*)₄]ₚ(Va)

where
M_{β} is a transition metal of groups 8 to 10 of the Periodic Table of the Elements having a formal charge of −1 or −2,
L* is a ligand selected from among PR₃, P(OR)₃, NR₃, SR₂, OR₂, CO, NO, R—CN, R—NO₂, (RO)(R′O)C═O, (R)(R′)C═O, (R)C═O(OR′),
M_{α} is a metal of group 1 or 2 of the Periodic Table of the Elements, Zn or Hg, bis(triarylphosphine)iminium, imidazolium, pyridinium, pyrrolidinium, guanidinium, isouronium, trityl or T(R)₄ where
T is N, P or As,
R, R′ are each, independently of one another, hydrogen, alkyl, aryl, alkaryl or aralkyl,
n, m are each 1 or 2,
p is n·m in the case of a formal charge on M_{β} of −1 or is n·m/2 in the case of a formal charge on M_{β} of −2.

Possible radicals R or R′ are, for example, hydrogen, straight-chain or branched C₁-C₁₀-alkyl such as methyl, ethyl, n- or i-propyl, n-, i- or t-butyl or n- or i-pentyl, C₆-C₁₄-aryl such as phenyl or naphthyl or alkylaryl having from 1 to 10 carbon atoms in the alkyl part and from 6 to 14 carbon atoms in the aryl part, e.g. benzyl. Suitable aromatic radicals also comprise heterocycles and may be, for example, 5- or 6-membered monocyclic compounds such as pyridyl and phenyl and also fused systems such as anthracene.

Possible metallic cations M_{α} are, inter alia, alkali metal and alkaline earth metal cations. Preference is given to employing lithium, sodium, potassium and/or cesium.

Among nonmetallic cations M_{α} preference is given to tetraphenyl-, tetramethyl-, tetraethyl- and tetra-n-butylammonium, -phosphonium and -arsenium, bis(triaryl-phosphine)iminium, imidazolium, pyridinium, pyrrolidinium, guanidinium or isouronium. Particularly suitable aryl radicals in the bis(triarylphosphine)iminium cation are phenyl and naphthyl, with preference being given to bis(triphenylphosphine)iminium.

The anionic complexes Va are preferably selected from the group consisting of Li[Co(CO)₄], Na[Co(CO)₄], K[Co(CO)₄], Cs[Co(CO)₄], (R₄N)[Co(CO)₄], (R₄P)[Co(CO)₄], (R₄As)[Co(CO)₄], (PPN)[Co(CO)₄], Li[Rh(CO)₄], Na[Rh(CO)₄], K[Rh(CO)₄], Cs[Rh(CO)₄], (R₄N)[Rh(CO)₄], (R₄P)[Rh(CO)₄], (R₄As)[Rh(CO)₄], (PPN)[Rh(CO)₄], Li[Ir(CO)₄], Na[Ir(CO)₄], K[Ir(CO)₄], Cs[Ir(CO)₄], (R₄N)[Ir(CO(₄], (RᵣP)[Ir(CO)₄], (R₄As)[Ir(CO)₄], (PPN)[Ir(CO)₄], Li₂[Fe(CO)₄], Na₂[Fe(CO)₄], K₂[Fe(CO)₄], (R₄N)₂[Fe(CO)₄], (R₄P)₂[Fe(CO)₄], (R₄As)₂[Fe(CO)₄], (PPN)₂[Fe(CO)₄], (PPN)[HFe(CO)₄] and (PPN)₂[Fe₂(CO)₈], where R is methyl, ethyl, n- or i-propyl, n-, i- or t-butyl, phenyl or benzyl.

Among the anionic complexes Va comprising cobalt in the oxidation state −1, particular preference is given to tetraphenylphosphonium, tetraphenylarsenium, tetraphenylammonium, tetraethylphosphonium, tetraethylarsenium, tetraethylammonium, imidazolium, pyridinium, pyrrolidinium, guanidinium and isouronium tetracarbonylcobaltate, and also sodium tetracarbonylcobaltate. Na[Co(CO)₄] is particularly preferred.

The preparation of the anionic complexes is known to those skilled in the art. Suitable preparative methods are described, for example, in F. G. Stone, E. W. Abel and G. Wilkinson, “Comprehensive Organometallic Chemistry—The Synthesis, Reactions and Structures of Organometallic Compounds”, Pergamon, Oxford, 1982, and F. G. Stone, E. W. Abel and G. Wilkinson, “Comprehensive Organometallic Chemistry II—A Review of the Literature 1982-1994”, Pergamon Press, Oxford, for example in Vol. 8. Moreover, such complexes are also commercially available.

Of course, it is also possible to use mixtures of various uncharged and/or anionic complexes V. The complexes can be mononuclear or multinuclear.

The amount of complexes V in the reaction mixture is usually in the range from 0.01 to 10 000 mol %, preferably from 0.1 to 100 mol %, particularly preferably from 0.2 to 10 mol %, calculated as the sum of all uncharged and anionic transition metal complexes V and based on the amount of epoxide used.

The complexes V can also be used in the form of an ionic liquid. In this case, they simultaneously serve as solvent and reaction medium. The amount of complex V based on the amount of lactone used can here be present in a large excess. These ionic liquids are commercially available, e.g. 1-butyl-3-methylimidazolium tetracarbonylcobaltate.

### EXAMPLES

The chemicals used come from Fluka, Aldrich or Merck, unless indicated otherwise, and were used without further purification. The solvents were dried over molecular sieves and in each case degassed and saturated with N₂ before use. The β-butyrolactone was dried over calcium hydride, distilled and stored over molecular sieves.
Salen Cr1: 1,2-phenylenediamino-N,N′-bis(3,5-di-t-butylsalicylidene)]chromium(III) chloride

The salen ligand was synthesized by condensation of 20.3 mmol of ortho-phenylenediamine and 42.6 mmol of 3,5-di-tert-butyl-2-hydroxybenzaldehyde in ethanol, as described in WO 00/09463.

9.7 mmol of ligand and 11.6 mmol of chromium(II) chloride were stirred in 120 ml of tetrahydrofuran at room temperature under a nitrogen atmosphere for 5 hours. The mixture was subsequently stirred for 12 hours under an air atmosphere. After addition of 23.2 mmol of 2,6-lutidine, the mixture was stirred for another 3 hours under a nitrogen atmosphere. 450 ml of tert-butyl methyl ether were subsequently added, and the mixture was shaken three times with 150 ml of saturated aqueous ammonium chloride solution and twice with saturated aqueous NaCl solution. The organic phase was dried over sodium sulfate, the sodium sulfate was filtered and the solvent was taken off and the product was dried under reduced pressure.
Salen Cr2: 4,5-dichloro-1,2-phenylenediamino-N,N′-bis(3,5-di-t-butylsalicylidene)]chromium(III) chloride

The salen ligand was synthesized by condensation of 5 mmol of 4,5-dichloro-ortho-phenylenediamine and 10.4 mmol of 3,5-di-tert-butyl-2-hydroxybenzaldehyde in 50 ml of ethanol.

2.5 mmol of ligand and 3 mmol of chromium(II) chloride were stirred in 50 ml of THF at room temperature under a nitrogen atmosphere for 5 hours. The mixture was subsequently stirred for 12 hours under an air atmosphere. After addition of 6 mmol of 2,6-lutidine, the mixture was stirred for another 3 hours under a nitrogen atmosphere. 100 ml of tert-butyl methyl ether were subsequently added, and the mixture was shaken three times with 25 ml of saturated aqueous ammonium chloride solution and twice with saturated aqueous NaCl solution. The organic phase is dried over sodium sulfate, the sodium sulfate is filtered and the solvent is taken off and the product is dried under reduced pressure.

The isotacticity was determined by means of ¹³C-NMR spectroscopy. This was carried out using the method described in Macromolecules 1989, 22, 1656, and the two peaks of the isotactic and syndiotactic diads in the carbonyl range at 169 ppm were integrated.

Polymerization Tests:

### Example 1

15.5 ml of racemic β-butyrolactone were placed in a 250 ml glass flask. 39 mg of salen Cr1 were subsequently added and the mixture was heated to 100° C. After 20 hours, the mixture was cooled and a sample was taken for the determination of the conversion by means of ¹H-NMR spectroscopy. The polymer was subsequently precipitated in hexane/ether and dried. The dried polymer was characterized by means of ¹³C-NMR spectroscopy.

The conversion was 92% and the proportion of isotactic diads was 69%.

Polymer Characterization:

The thermal properties of the polymer from Example 1 were examined by means of DSC (differential scanning calorimetry). The heating rate was 20°/min. In the first heating, a glass transition at −10° C. and melting points at 121 and 141° C. were found. In a second heating after cooling to −30° C. at 20°/min, a glass transition at 0° C. and melting points at 112 and 142° C. were found.

The molecular weight of the polymer was determined by means of size exclusion chromatography in hexafluoroisopropanol (column temperature: 40° C., calibration using PMMA standard). This gave a number average of 20 000 dalton and a weight average of 136 000 dalton.

The polymer was fractionated by precipitation in methanol and the melting point of the precipitated polymer was determined. The heating rate was 20°/min. In the first heating, broad melting points at 117 and 145° C. were found. In a second heating after cooling to −30° C. at 20°/min, a glass transition at 3° C. and melting points at 123 and 146° C. were found.

### Example 2

15.5 ml of a mixture of 60% of R— and 40% of S-β-butyrolactone were placed in a 250 ml glass flask. 118 mg of salen Cr1 were subsequently added and the mixture was heated to 100° C. After 16 hours, the mixture was cooled and a sample was taken for the determination of the conversion by means of ¹H-NMR spectroscopy. The polymer was subsequently precipitated in hexane/ether and dried. The dried polymer was characterized by means of ¹³C-NMR spectroscopy.

The conversion was 60% and the proportion of isotactic diads was 74%.

### Example 3

15.5 ml of racemic β-butyrolactone were placed in a 250 ml glass flask. 118 mg of salen Cr1 and 5 mg of tetrabutylammonium chloride were subsequently added and the mixture was heated to 100° C. After 16 hours, the mixture was cooled and a sample was taken for the determination of the conversion by means of ¹H-NMR spectroscopy. The polymer was subsequently precipitated in hexane/ether and dried. The dried polymer was characterized by means of ¹³C-NMR spectroscopy.

The conversion was 85% and the proportion of isotactic diads was 64%.

### Example 4

15.5 ml of racemic β-butyrolactone were placed in a 250 ml glass flask. 131 mg of salen Cr2 were subsequently added and the mixture was heated to 100° C. After 20 hours, the mixture was cooled and a sample was taken for the determination of the conversion by means of ¹H-NMR spectroscopy. The polymer was subsequently precipitated in hexane/ether and dried.

The conversion was 82% and the proportion of isotactic diads was 57%.

The thermal properties of the polymer from Example 4 were examined by means of DSC (differential scanning calorimetry). The heating rate was 20°/min. In the first heating, a glass transition at 5° C. and broad melting points at 91 and 126° C. were found. In a second heating after cooling to −30° C. at 20°/min, a glass transition at 6° C. and a broad melting range having a maximum at 135° C. were found.

The molecular weight of the polymer was determined by means of size exclusion chromatography in hexafluoroisopropanol (column temperature: 40° C., calibration using PMMA standard). This gave a number average of 132 000 dalton and a weight average of 660 000 dalton.

1-8. (canceled)

9. A process for preparing polyhydroxyalkanoates by polymerization of lactones of the general formula I, 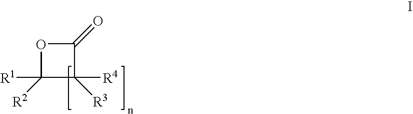
wherein the substituents and the index n have the following meanings:
n is from 1 to 4;
R¹, R², R³, R⁴ are each, independently of one another, hydrogen, C₁-C₁₂-alkyl, C₂-C₈-alkenyl, C₃-C₈-cycloalkyl, C₆-C₁₂-aryl, C₆-C₁₂-aryl-C₁-C₃-alkyl, halogen, nitro, C₁-C₆-alkoxy, C₆-C₁₂-aryloxy, amino, C₁-C₆-alkylamino, di(C₁-C₆-alkyl)amino, di(C₁-C₆-alkyl)phosphino, C₁-C₆-alkylsulfinyl, C₁-C₆-alkylsulfonyl; and
two radicals R¹ to R⁴ are located on adjacent ring carbons together form C₁-C₅-alkylene; wherein R¹ to R⁴ may in turn be substituted by R^{x} and R^{x} represents from one to three radicals selected from among halogen, cyano, nitro, C₁-C₆-alkoxy, C₁-C₆-alkylthio, di(C₁-C₆-alkyl)amino, C₆-C₁₂-aryloxy, C₆-C₁₂-aryl-C₁-C₃-alkoxy, C₁-C₆-alkoxycarbonyl, C₆-C₁₂-aryloxycarbonyl, C₆-C₁₂-aryl-C₁-C₃-alkoxycarbonyl, C₁-C₆-alkylcarbonyl, C₆-C₁₂-arylcarbonyl, C₆-C₁₂-aryl-C₁-C₃-alkylcarbonyl, C₁-C₆-alkylsulfinyl, C₆-C₁₂-arylsulfinyl, C₆-C₁₂-aryl-C₁-C₃-alkylsulfinyl, C₁-C₆-alkylsulfonyl, C₆-C₁₂-arylsulfonyl, C₆-C₁₂-aryl-C₁-C₃-alkylsulfonyl;
i) in the presence of at least one catalyst of the formula (II) L_{I}M^{a}X^{a}ₘ, wherein the substituents and indices have the following meanings:
M^{a} is chromium;
X^{a} is an anion selected from the group consisting of halide, sulfate, sulfite, nitrate, nitrite, carboxylate, hydroxide, alkoxide, thiolate, phosphate, sulfonate, borate, phenoxide, antimonate, cobaltate and ferrate;
L is a ligand selected from the group consisting of imine, amine, phosphane, ylide, carbonyl, nitrile, ester, ether, sulfide, amide, cyclopentadienyl, ansa compounds, alkoxide, phenoxide, carboxylate, thiolate, imide, sulfonate, porphyrin, phthalocyanine, oxazoline, salen and Schiff base compounds, with different ligands L also being able to be joined to one another and the ligands L being able to be substituted; and
l, m are integers from 1 to 10 which are selected so that the compound of the general formula II is uncharged;
ii) optionally in the presence of an activator compound B selected from among unsubstituted or substituted pyridines, imidazoles, triazoles, carbenes, phosphines and ionic compounds of the general formula (III) X^{b}ₒYₚ, wherein the substituents and indices have the following meanings:
X^{b} is a cation selected from the group consisting of H⁺, Na⁺, K⁺, Li⁺, Mg²⁺, Ca²⁺, Al³⁺, NR₄⁺, pyridinium, imidazolium, PR₄⁺, AsR₄⁺ and N[PR₃)]₂⁺, where R is hydrogen, C₁-C₆-alkyl or C₆-C₁₂-aryl;
Y is an anion selected from the group consisting of halide, carboxylate, dicarboxylate, tricarboxylate, polycarboxylate, sulfonate, sulfonyl, sulfate, sulfinylate, phosphate, phosphite, hydroxide, alkoxide, dialkoxide, trialkoxide, polyalkoxide, thiolate, acyl, carbonate, carbamate, antimonate and borate; and
o, p are integers from 1 to 10 000 which are selected so that the compound of the general formula III is uncharged; and
iii) optionally in the presence of a Lewis acid C of the general formula (IV) L_{q}M^{c}X^{c}ᵣ, where the substituents and indices have the following meanings:
M^{c} is a metal selected from the group consisting of Mg, Ca, Sc, Y, rare earth elements, Ti, V, Mn, Fe, Co, Ni, Cu, Zn, Al, Ga, Zr, Nb, Ru, Rh, Pd, Ag, Cd, In, Hf, Ta, Re, Os, Ir, Pt, Au, Hg, Tl and Pb;
X^{c} is an anion selected from the group consisting of halide, sulfate, sulfite, nitrate, nitrite, imide, carboxylate, sulfide, phosphate, sulfonate, borate, hydroxide, alkoxide, phenoxide, antimonate, cobaltate and ferrate;
L is a ligand selected from the group consisting of imine, amine, phosphane, ylide, carbonyl, nitrile, ester, ether, sulfide, amide, cyclopentadienyl, ansa compounds, alkoxide, phenoxide, carboxylate, thiolate, imide, sulfonate, porphyrin, phthalocyanine, oxazoline, salen and Schiff base compounds, with different ligands L also being able to be joined to one another and the ligands L being able to be substituted; and
q, r are integers from 1 to 10 which are selected so that the compound of the general formula IV is uncharged.

10. The process according to claim 9, wherein a chromium(III)-salen complex of the formula II is used as a catalyst.

11. The process according to claim 9 by polymerization of chiral lactones of the formula I.

12. The process according to claim 11 by polymerization of a racemate of chiral lactones.

13. The process according to claim 11 by polymerization of β-butyrolactone.

14. The process according to claim 11, wherein a chromium(III)-salen complex of the formula II is used as a catalyst.

15. The process according to claim 10 by polymerization of chiral lactones of the formula I.

16. The process according to claim 15 by polymerization of a racemate of chiral lactones.

17. The process according to claim 15 by polymerization of β-butyrolactone.

18. The process according to claim 15, wherein a chromium(III)-salen complex of the formula II is used as a catalyst.

19. The process according to claim 9, wherein the lactone is prepared from oxirane and CO in the presence of a carbonylation catalyst V in a preceding step and is converted into the polyhydroxyalkanoate without intermediate isolation of the lactone.

20. The process according to claim 19, wherein the lactone is prepared in the additional presence of one or more compounds selected from the group consisting of catalyst II, activator compound III and Lewis acid IV, as defined in claim 9.

21. The process according to claim 10, wherein the lactone is prepared from oxirane and CO in the presence of a carbonylation catalyst V in a preceding step and is converted into the polyhydroxyalkanoate without intermediate isolation of the lactone.

22. The process according to claim 21, wherein the lactone is prepared in the additional presence of one or more compounds selected from the group consisting of catalyst II, activator compound III and Lewis acid IV, as defined in claim 9.

23. The process according to claim 11, wherein the lactone is prepared from oxirane and CO in the presence of a carbonylation catalyst V in a preceding step and is converted into the polyhydroxyalkanoate without intermediate isolation of the lactone.

24. The process according to claim 23, wherein the lactone is prepared in the additional presence of one or more compounds selected from the group consisting of catalyst II, activator compound III and Lewis acid IV, as defined in claim 9.

25. The process according to claim 12, wherein the lactone is prepared from oxirane and CO in the presence of a carbonylation catalyst V in a preceding step and is converted into the polyhydroxyalkanoate without intermediate isolation of the lactone.

26. The process according to claim 25, wherein the lactone is prepared in the additional presence of one or more compounds selected from the group consisting of catalyst II, activator compound III and Lewis acid IV, as defined in claim 9.

27. The process according to claim 13, wherein the lactone is prepared from oxirane and CO in the presence of a carbonylation catalyst V in a preceding step and is converted into the polyhydroxyalkanoate without intermediate isolation of the lactone.

28. The process according to claim 27, wherein the lactone is prepared in the additional presence of one or more compounds selected from the group consisting of catalyst II, activator compound III and Lewis acid IV, as defined in claim 9.
